(19) United States
(12) Patent Application Publication
(10) Pub. No.: US 2006/0199773 A1
(43) Pub. Date: Sep. 7, 2006

(54) Crystalline forms of (1R,2S)-N-[(1,1-dimethylethoxy)carbonyl]-3-methyl-L-valyl-(4R)-4-[(6-methoxy-1-isoquinolinyl)oxy]-L-prolyl-1-amino-N-(cyclopropylsulfonyl)-2-ethenyl-cyclopropanecarboxamide, monopotassium salt

(76) Inventors: Justin B. Sausker, Middletown, CT (US); Paul Michael Scola, Glastonbury, CT (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/390,823

(22) Filed: Mar. 28, 2006

Related U.S. Application Data
(63) Continuation-in-part of application No. 11/295,914, filed on Dec. 7, 2005.
Application No. 11/295,914 is a continuation of application No. 10/441,657, filed on May 20, 2003, now Pat. No. 6,995,174.
(60) Provisional application No. 60/382,055, filed on May 20, 2002.

(51) Int. Cl. A61K 38/05; C07K 5/06

(52) U.S. Cl. 514/18; 514/309; 530/331

(57) ABSTRACT

The present disclosure generally relates to crystalline forms of (1R,2S)-N-[(1,1 -dimethylethoxy)carbonyl]-3-methyl-L-valyl-(4R)-4-[(6-methoxy-1-isoquinolinyl)oxy]-L-prolyl-1-amino-N-(cyclopropylsulfonyl)-2-ethenyl-cyclopropanecarboxamide. The present disclosure also generally relates to a pharmaceutical composition comprising a crystalline form, as well of methods of using a crystalline form in the treatment of Hepatitis C and methods for obtaining such crystalline form.

### CROSS REFERENCE TO RELATED APPLICATIONS

This application is a continuation-in-part of U.S. application Ser. No. 11/295,914, filed Dec. 7, 2005, which is a continuation of U.S. application Ser. No. 10/441,657, filed May 20, 2003, now U.S. Pat. No. 6,995,174, which claims priority to U.S. Provisional Application Ser. No. 60/382,055, filed May 20, 2002.

### FIELD OF THE DISCLOSURE

The present disclosure generally relates to a crystalline form of (1R,2S)-N-[(1,1 -dimethylethoxy)carbonyl]-3-methyl-L-valyl-(4R)-4-[(6-methoxy-1-isoquinolinyl)oxy]-L-prolyl-1-amino-N-(cyclopropylsulfonyl)-2-ethenyl-cyclopropanecarboxamide, monopotassium salt. The present disclosure also generally relates to a pharmaceutical composition comprising a crystalline form, as well of methods of using a crystalline form in the treatment of Hepatitis C virus (HCV) and methods for obtaining such crystalline form.

### BACKGROUND OF THE DISCLOSURE

Hepatitis C virus (HCV) is a major human pathogen, infecting an estimated 170 million persons worldwide—roughly five times the number infected by human immunodeficiency virus type 1. A substantial fraction of these HCV infected individuals develop serious progressive liver disease, including cirrhosis and hepatocellular carcinoma.

Presently, the most effective HCV therapy employs a combination of alpha-interferon and ribavirin, leading to sustained efficacy in 40 percent of patients. Recent clinical results demonstrate that pegylated alpha-interferon is superior to unmodified alpha-interferon as monotherapy. However, even with experimental therapeutic regimens involving combinations of pegylated alpha-interferon and ribavirin, a substantial fraction of patients do not have a sustained reduction in viral load. Thus, there is a clear and unmet need to develop effective therapeutics for treatment of HCV infection.

The compound (1R,2S)-N-[(1,1-dimethylethoxy)carbonyl]-3-methyl-L-valyl-(4R)-4-[(6-methoxy-1-isoquinolinyl)oxy]-L-prolyl-1-amino-N-(cyclopropylsulfonyl)-2-ethenyl-cyclopropanecarboxamide, described in US20040106559, is useful for the treatment of HCV infection. Due to the low aqueous solubility of this compound, formulation of the compound presents a significant challenge. It has been found that the potassium salt, represented by formula (I) and herein referred to as Compound (I), offers improved aqueous solubility. This compound has also been described in US20040106559.

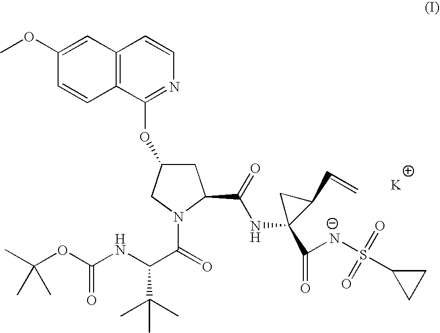

### SUMMARY OF THE DISCLOSURE

In a first aspect the present disclosure provides a crystalline form of (1R,2S)-N-[(1,1-dimethylethoxy)carbonyl]-3-methyl-L-valyl-(4R)-4-[(6-methoxy-1-isoquinolinyl)oxy]-L-prolyl-1-amino-N-(cyclopropylsulfonyl)-2-ethenyl-cyclopropanecarboxamide or compound (I) 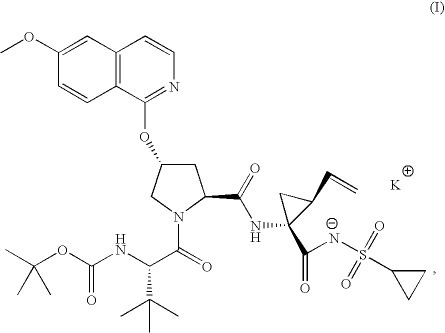
comprising Form N-1.

In one embodiment of the first aspect the present disclosure provides the crystalline form of (1R,2S)-N-[(1,1-dimethylethoxy)carbonyl]-3-methyl-L-valyl-(4R)-4-[(6-methoxy-1-isoquinolinyl)oxy]-L-prolyl-1-amino-N-(cyclopropylsulfonyl)-2-ethenyl-cyclopropanecarboxamide or compound (I) consisting essentially of Form N-1.

In another embodiment of the first aspect the present disclosure provides the crystalline form of (1 R,2S)-N-[(1,1-dimethylethoxy)carbonyl]-3-methyl-L-valyl-(4R)-4-[(6-methoxy-1-isoquinolinyl)oxy]-L-prolyl-1-amino-N-(cyclopropylsulfonyl)-2-ethenyl-cyclopropanecarboxamide or compound (I) comprising Form N-1 wherein said Form N-1 has a purity of at least 90 weight percent. In another embodiment said Form N-1 has a purity of at least 95 weight percent. In another embodiment said Form N-1 has a purity of at least 99 weight percent.

In another embodiment of the first aspect the present disclosure provides the crystalline form of (1R,2S)-N-[(1,1-dimethylethoxy)carbonyl]-3-methyl-L-valyl-(4R)-4-[(6-methoxy-1-isoquinolinyl)oxy]-L-prolyl-1-amino-N-(cyclopropylsulfonyl)-2-ethenyl-cyclopropanecarboxamide or compound (I) comprising Form N-1 wherein the crystalline form is characterized by unit cell parameters substantially equal to the following:
Cell dimensions: a=6.2239 Å
b=20.9360 Å
c=29.1860 Å
α=90 degrees
β=90 degrees
γ=90 degrees
Space group P2₁2₁2₁
Molecules/unit cell 4
   wherein measurement of said crystalline form is at a temperature between about 20° C. to about 25° C.;

In another embodiment of the first aspect the present disclosure provides the crystalline form of (1R,2S)-N-[(1,1-dimethylethoxy)carbonyl]-3-methyl-L-valyl-(4R)-4-[(6-methoxy-1-isoquinolinyl)oxy]-L-prolyl-1-amino-N-(cyclopropylsulfonyl)-2-ethenyl-cyclopropanecarboxamide or compound (I) comprising Form N-1 wherein the crystalline form is characterized by fractional atomic coordinates substantially as listed in Table 3.

In another embodiment of the first aspect the present disclosure provides the crystalline form of (1R,2S)-N-[(1,1-dimethylethoxy)carbonyl]-3-methyl-L-valyl-(4R)-4-[(6-methoxy-1-isoquinolinyl)oxy]-L-prolyl-1-amino-N-(cyclopropylsulfonyl)-2-ethenyl-cyclopropanecarboxamide or compound (I) comprising Form N-1wherein the crystalline form is characterized by a powder X-Ray diffraction pattern comprising four or more 2θ values (CuKα λ=1.5418 Å) selected from the group consisting of 5.2, 6.1, 7.4, 8.4, 9.0, 10.0, 10.4, 12.1, 16.0, and 16.8 at a temperature between about 20° C. and about 25° C. In another embodiment the crystalline form is further characterized by a powder X-Ray diffraction pattern comprising five or more 2θ values (CuKα λ=1.5418 Å) selected from the group consisting of 5.2, 6.1, 7.4, 8.4, 9.0, 10.0, 10.4, 12.1, 16.0, and 16.8 at a temperature between about 20° C. and about 25° C.

In another embodiment of the first aspect the present disclosure provides the crystalline form of (1R,2S)-N-[(1,1-dimethylethoxy)carbonyl]-3-methyl-L-valyl-(4R)-4-[(6-methoxy-1-isoquinolinyl)oxy]-L-prolyl-1-amino-N-(cyclopropylsulfonyl)-2-ethenyl-cyclopropanecarboxamide or compound (I) comprising Form N-1 wherein the crystalline form is characterized by one or more of the following:
a) a unit cell with parameters substantially equal to the following:
   Cell dimensions: a =6.2239 Å
   b=20.9360 Å
   c=29.1860Å
   α=90 degrees
   β=90 degrees
   γ=90 degrees
   Space group P2₁2₁2₁
   Molecules/unit cell 4
      wherein measurement of said crystalline form is at a temperature between about 20° C. and about 25° C.;
b) a powder X-Ray diffraction pattern comprising four or more 2θ values (CuKαλ=1.5418 Å) selected from the group consisting of 5.2, 6.1, 7.4, 8.4, 9.0, 10.0, 10.4, 12.1, 16.0, and 16.8 at a temperature between about 20° C. and about 25° C.; and/or
c) a melting point in the range of about 252° C. to about 262° C.

In a second aspect the present disclosure provides a pharmaceutical composition comprising a crystalline form of (1R,2S)-N-[(1,1-dimethylethoxy)carbonyl]-3-methyl-L-valyl-(4R)-4-[(6-methoxy-1-isoquinolinyl)oxy]-L-prolyl-1-amino-N-(cyclopropylsulfonyl)-2-ethenyl-cyclopropanecarboxamide or compound (I) comprising Form N-1 and a pharmaceutically acceptable carrier or diluent.

In one embodiment of the second aspect the present disclosure provides a pharmaceutical composition comprising a crystalline form of (1R,2S)-N-[(1,1-dimethylethoxy)carbonyl]-3-methyl-L-valyl-(4R)-4-[(6-methoxy-1-isoquinolinyl)oxy]-L-prolyl-1-amino-N-(cyclopropylsulfonyl)-2-ethenyl-cyclopropanecarboxamide or compound (I) comprising Form N-1 and a pharmaceutically acceptable carrier or diluent wherein Form N-1 has a purity of at least 90 weight percent. In another embodiment Form N-1 has a purity of at least 95 weight percent. In another embodiment Form N-1 has a purity of at least 99 weight percent.

In another embodiment of the second aspect the present disclosure provides a pharmaceutical composition comprising a crystalline form of (1R,2S)-N-[(1,1-dimethylethoxy)carbonyl]-3-methyl-L-valyl-(4R)-4-[(6-methoxy-1-isoquinolinyl)oxy]-L-prolyl-1-amino-N-(cyclopropylsulfonyl)-2-ethenyl-cyclopropanecarboxamide or compound (I) comprising Form N-1 and a pharmaceutically acceptable carrier or diluent in combination with a second compound having anti-HCV activity.

In another embodiment of the second aspect the present disclosure provides a pharmaceutical composition comprising a crystalline form of (1R,2S)-N-[(1,1-dimethylethoxy)carbonyl]-3-methyl-L-valyl-(4R)-4-[(6-methoxy-1-isoquinolinyl)oxy]-L-prolyl-1-amino-N-(cyclopropylsulfonyl)-2-ethenyl-cyclopropanecarboxamide or compound (I) comprising Form N-1 and a pharmaceutically acceptable carrier or diluent in combination with a second compound having anti-HCV activity wherein Form N-1 has a purity of at least 90 weight percent. In another embodiment Form N-1 has a purity of at least 95 weight percent. In another embodiment Form N-1 has a purity of at least 99 weight percent.

In another embodiment of the second aspect the present disclosure provides a pharmaceutical composition comprising a crystalline form of (1R,2S)-N-[(1,1-dimethylethoxy)carbonyl]-3-methyl-L-valyl-(4R)-4-[(6-methoxy-1-isoquinolinyl)oxy]-L-prolyl-1-amino-N-(cyclopropylsulfonyl)-2-ethenyl-cyclopropanecarboxamide or compound (I) comprising Form N-1 and a pharmaceutically acceptable carrier or diluent in combination with a second compound having anti-HCV activity wherein the second compound having anti-HCV activity is an interferon. In another embodiment the interferon is selected from interferon alpha 2B, pegylated interferon alpha, consensus interferon, interferon alpha 2A, and lymphoblastiod interferon tau.

In another embodiment of the second aspect the present disclosure provides a pharmaceutical composition comprising a crystalline form of (1R,2S)-N-[(1,1-dimethylethoxy)carbonyl]-3-methyl-L-valyl-(4R)-4-[(6-methoxy-1-isoquinolinyl)oxy]-L-prolyl-1-amino-N-(cyclopropylsulfonyl)-2-ethenyl-cyclopropanecarboxamide or compound (I) comprising Form N-1 and a pharmaceutically acceptable carrier or diluent in combination with a second compound having anti-HCV activity wherein the second compound having anti-HCV activity is selected from interleukin 2, interleukin 6, interleukin 12, a compound that enhances the development of a type 1 helper T cell response, interfering RNA, anti-sense RNA, Imiqimod, ribavirin, an inosine 5′-monophospate dehydrogenase inhibitor, amantadine, and rimantadine.

In a third aspect the present disclosure provides a method of treating HCV infection in a mammal comprising administering to the mammal a therapeutically-effective amount of the crystalline form of (1R,2S)-N-[(1,1-dimethylethoxy)carbonyl]-3-methyl-L-valyl-(4R)-4-[(6-methoxy-1-isoquinolinyl)oxy]-L-prolyl-1-amino-N-(cyclopropylsulfonyl)-2-ethenyl-cyclopropanecarboxamide or compound (I) 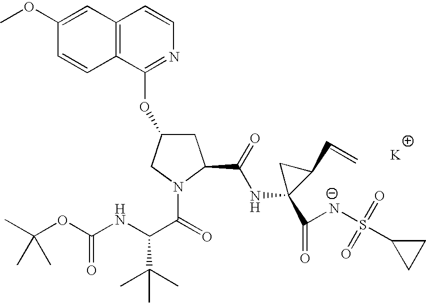
comprising Form N-1.

In one embodiment of the third aspect the present disclosure provides a method of treating HCV infection in a mammal comprising administering to the mammal a therapeutically-effective amount of the crystalline form of (1R,2S)-N-[(1,1-dimethylethoxy)carbonyl]-3-methyl-L-valyl-(4R)-4-[(6-methoxy-1-isoquinolinyl)oxy]-L-prolyl-1-amino-N-(cyclopropylsulfonyl)-2-ethenyl-cyclopropanecarboxamide or compound (I) comprising Form N-1 wherein Form N-1 has a purity of at least 90 weight percent. In another embodiment Form N-1 has a purity of at least 95 weight percent. In another embodiment Form N-1 has a purity of at least 99 weight percent.

In another embodiment of the third aspect the present disclosure provides a method of treating HCV infection in a mammal comprising administering to the mammal a therapeutically-effective amount of the crystalline form of (1R,2S)-N-[(1,1-dimethylethoxy)carbonyl]-3-methyl-L-valyl-(4R)-4-[(6-methoxy-1-isoquinolinyl)oxy]-L-prolyl-1-amino-N-(cyclopropylsulfonyl)-2-ethenyl-cyclopropanecarboxamide or compound (I) comprising Form N-1 wherein the mammal is a human.

In a fourth aspect the present disclosure provides a composition comprising at least 90 weight percent of the crystalline form of (1R,2S)-N-[(1,1-dimethylethoxy)carbonyl]-3-methyl-L-valyl-(4R)-4-[(6-methoxy-1-isoquinolinyl)oxy]-L-prolyl-1-amino-N-(cyclopropylsulfonyl)-2-ethenyl-cyclopropanecarboxamide or compound (I) comprising Form N-1, based the weight of the composition.

### BRIEF DESCRIPTION OF THE FIGURES

FIG. 1 illustrates experimental and simulated powdered X-Ray diffraction patterns (CuKα λ=1.5418 Å at T 293 K) of the N-1 crystalline form of Compound (I).

FIG. 2 illustrates the differential scanning calorimetry pattern of the N-1 crystalline form of Compound (I).

FIG. 3 illustrates the thermogravimetric analysis pattern of the N-1 crystalline form of Compound (I).

### DETAILED DESCRIPTION

The disclosure relates to a crystalline form of Compound (I). 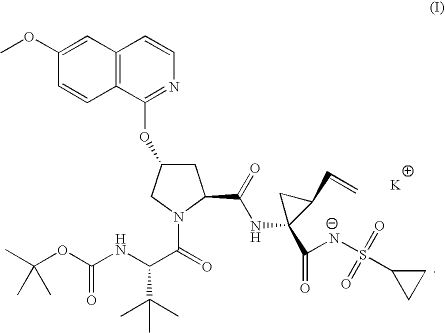
The name used herein to characterize this form, i.e. “N-1”, should not be considered limiting with respect to any other substance possessing similar or identical physical and chemical characteristics, but rather it should be understood that this designation is a mere identifier that should be interpreted according to the characterization information also presented herein.
Definitions

As used herein “polymorph” refers to crystalline forms having the same chemical composition but different spatial arrangements of the molecules, atoms, and/or ions forming the crystal.

The term “pharmaceutically acceptable,” as used herein, refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable beneift/risk ratio.

The term “therapeutically effective amount,” as used herein, is intended to include an amount of the crystalline forms of Compound (I) that is effective when administered alone or in combination to treat Hepatitis C. The crystalline forms of Compound (I) and pharmaceutical compositions thereof may be useful in treating Hepatitis C. If Compound (I) is used in combination with another medication, the combination of compounds described herein may result in a synergistic combination. Synergy, as described for example by Chou and Talalay, Adv. Enzyme Regul. 1984, 22, 27-55, occurs when the effect of the compounds when administered in combination is greater than the effect of the compounds when administered alone as single agents.

The term “treating” refers to: (i) preventing a disease, disorder or condition from occurring in a patient which may be predisposed to the disease, disorder and/or condition but has not yet been diagnosed as having it; (ii) inhibiting the disease, disorder or condition, i.e., arresting its development; and/or (iii) relieving the disease, disorder or condition, i.e., causing regression of the disease, disorder and/or condition.

In one embodiment the disclosure provides a crystalline form of Compound (I). This crystalline form of Compound (I) may be employed in pharmaceutical compositions which may optionally include one or more other components selected, for example, from the group consisting of excipients, carriers, and one of other active pharmaceutical ingredients active chemical entities of different molecular structure.

Preferably, the crystalline form has phase homogeneity indicated by less than 10 percent, preferably less than 5 percent, and more preferably less than 2 percent of the total peak area in the experimentally measured PXRD pattern arising from the extra peaks that are absent from the simulated PXRD pattern. Most preferred is a crystalline form having phase homogeneity with less than 1 percent of the total peak area in the experimentally measured PXRD pattern arising from the extra peaks that are absent from the simulated PXRD pattern.

In one embodiment, a composition is provided consisting essentially of the crystalline form N-1 of Compound (I). The composition of this embodiment may comprise at least 90 weight percent of the crystalline form N-1 of Compound (I), based on the weight of Compound (I) in the composition. The remaining material comprises other form(s) of the compound and/or reaction impuritis and/or processing impurities arising from its preparation.

Form N-1 is a neat, solvent-free form as shown by thermogravimetric analysis, elemental analysis, and Karl Fischer analysis. It takes up about 1.3 percent moisture between 0 and 90 percent relative humidity. Differential scanning calorimetry and thermogravimetric analysis show no evidence of conversions of the form or weight loss during heating before the melt/decomposition at ˜252° C. In addition, no conversion to a hydrate or to any other form was detected when form N-1 was kept in a water slurry at about 22° C. or at about 50° C. for 14 days.

The presence of reaction impurities and/or processing impurities may be determined by analytical techniques known in the art, such as, for example, chromatography, nuclear magnetic resonance spectroscopy, mass spectrometry, or infrared spectroscopy.

General Preparation of Crystalline Materials:

Crystalline forms may be prepared by a variety of methods, including for example, crystallization or recrystallization from a suitable solvent, sublimation, growth from a melt, solid state transformation from another phase, crystallization from a supercritical fluid, and jet spraying. Techniques for crystallization or recrystallization of crystalline forms from a solvent mixture include, for example, evaporation of the solvent, decreasing the temperature of the solvent mixture, crystal seeding a supersaturated solvent mixture of the molecule and/or salt, freeze drying the solvent mixture, and addition of antisolvents (countersolvents) to the solvent mixture. High throughput crystallization techniques may be employed to prepare crystalline forms including polymorphs. Crystals of drugs, including polymorphs, methods of preparation, and characterization of drug crystals are discussed in Solid-State Chemistry of Drugs, S. R. Byrn, R. R. Pfeiffer, and J. G. Stowell, 2^{nd} Edition, SSCI, West Lafayette, Ind. (1999).

For crystallization techniques that employ solvent, the choice of solvent or solvents is typically dependent upon one or more factors, such as solubility of the compound, crystallization technique, and vapor pressure of the solvent. Combinations of solvents may be employed, for example, the compound may be solubilized into a first solvent to afford a solution, followed by the addition of an antisolvent to decrease the solubility of the compound in the solution and to afford the formation of crystals. An antisolvent is a solvent in which the compound has low solubility.

In one method to prepare crystals, a compound is suspended and/or stirred in a suitable solvent to afford a slurry, which may be heated to promote dissolution. The term “slurry”, as used herein, means a saturated solution of the compound, which may also contain an additional amount of the compound to afford a heterogeneous mixture of the compound and a solvent at a given temperature.

Seed crystals may be added to any crystallization mixture to promote crystallization. Seeding may be employed to control growth of a particular polymorph or to control the particle size distribution of the crystalline product. Accordingly, calculation of the amount of seeds needed depends on the size of the seed available and the desired size of an average product particle as described, for example, in “Programmed Cooling of Batch Crystallizers,” J. W. Mullin and J. Nyvlt, Chemical Engineering Science, 1971,26, 369-377. In general, seeds of small size are needed to control effectively the growth of crystals in the batch. Seed of small size may be generated by sieving, milling, or micronizing of large crystals, or by micro-crystallization of solutions. Care should be taken that milling or micronizing of crystals does not result in any change in crystallinity of the desired crystal form (i.e., change to amorphous or to another polymorph).

A cooled crystallization mixture may be filtered under vacuum, and the isolated solids may be washed with a suitable solvent, such as cold recrystallization solvent, and dried under a nitrogen purge to afford the desired crystalline form. The isolated solids may be analyzed by a suitable spectroscopic or analytical technique, such as solid state nuclear magnetic resonance, differential scanning calorimetry, X-Ray powder diffraction, or the like, to assure formation of the preferred crystalline form of the product. The resulting crystalline form is typically produced in an amount of greater than about 70 weight percent isolated yield, preferably greater than 90 weight percent isolated yield, based on the weight of the compound originally employed in the crystallization procedure. The product may be co-milled or passed through a mesh screen to delump the product, if necessary.

Crystalline forms may be prepared directly from the reaction medium of the final process for preparing Compound (I). This may be achieved, for example, by employing in the final process step a solvent or a mixture of solvents from which Compound (I) may be crystallized. Alternatively, crystalline forms may be obtained by distillation or solvent addition techniques. Suitable solvents for this purpose include, for example, the aforementioned non-polar solvents and polar solvents, including protic polar solvents such as alcohols, and aprotic polar solvents such as ketones.

The presence of more than one polymorph in a sample may be determined by techniques such as powder X-Ray diffraction (PXRD) or solid state nuclear magnetic resonance spectroscopy. For example, the presence of extra peaks in the comparison of an experimentally measured PXRD pattern with a simulated PXRD pattern may indicate more than one polymorph in the sample. The simulated PXRD may be calculated from single crystal X-Ray data. see Smith, D. K., “A FORTRAN Program for Calculating X-Ray Powder Diffraction Patterns,” Lawrence Radiation Laboratory, Livermore, Calif. UCRL-7196 (April 1963).

Characterization:

Form N-1 of Compound (I) can be characterized using various techniques, the operation of which are well known to those of ordinary skill in the art. Examples of characterization methods include, but are not limited to, single crystal X-Ray diffraction, powder X-Ray diffraction (PXRD), simulated powder X-Ray patterns (Yin, S.; Scaringe, R. P.; DiMarco, J.; Galella, M. and Gougoutas, J. Z., American Pharmaceutical Review, 2003, 6, 2, 80), differential scanning calorimetry (DSC), solid-state ¹³C NMR (Earl, W. L. and Van der Hart, D. L., J. Magn. Reson., 1982, 48, 35-54), Raman spectroscopy, infrared spectroscopy, moisture sorption isotherms, and hot stage techniques.

The forms may be characterized and distinguished using single crystal X-Ray diffraction, which is based on unit cell measurements of a single crystal of form N-1. A detailed description of unit cells is provided in Stout & Jensen, X-Ray Structure Determination: A Practical Guide, Macmillan Co., New York (1968), Chapter 3, which is herein incorporated by reference. Alternatively, the unique arrangement of atoms in spatial relation within the crystalline lattice may be characterized according to the observed fractional atomic coordinates. Another means of characterizing the crystalline structure is by powder X-Ray diffraction analysis in which the diffraction profile is compared to a simulated profile representing pure powder material, both run at the same analytical temperature, and measurements for the subject form characterized as a series of 2θ values.

One of ordinary skill in the art will appreciate that an X-Ray diffraction pattern may be obtained with a measurement of error that is dependent upon the measurement conditions employed. In particular, it is generally known that intensities in an X-Ray diffraction pattern may fluctuate depending upon measurement conditions employed. It should be further understood that relative intensities may also vary depending upon experimental conditions, and, accordingly, the exact order of intensity should not be taken into account. Additionally, a measurement error of diffraction angle for a conventional X-Ray diffraction pattern is typically about 5 percent or less, and such degree of measurement error should be taken into account as pertaining to the aforementioned diffraction angles. Consequently, it is to be understood that the crystal forms of the present disclosure are not limited to the crystal forms that provide X-Ray diffraction patterns completely identical to the X-Ray diffraction patterns depicted in the accompanying Figures disclosed herein. Any crystal forms that provide X-Ray diffraction patterns substantially identical to those disclosed in the accompanying Figures fall within the scope of the present disclosure. The ability to ascertain substantial identities of X-Ray diffraction patters is within the purview of one of ordinary skill in the art.

Likewise, it is to be understood that any crystal forms that provide differential scanning calorimetry (DSC), thermogravimetric analysis (TGA), and/or moisture sorption isotherm patterns substantially identical to those disclosed in the accompanying Figures fall within the scope of the present disclosure. The ability to ascertain substantial identities of these patterns is within the purview of one of ordinary skill in the art.

Utility:

The N-1 form of Compound (I), alone or in combination with other compounds, can be used to treat HCV infection.

The present disclosure also provides compositions comprising a therapeutically effective amount of the N-1 form of Compound (I) and at least one pharmaceutically acceptable carrier.

The active ingredient, i.e., form N-1 of Compound (I), in such compositions typically comprises from 0.1 weight percent to 99.9 percent by weight of the composition, and often comprises from about 5 to 95 weight percent. In some cases, the pH of the formulation may be adjusted with pharmaceutically acceptable modifiers (such as calcium carbonate and magnesium oxide) to enhance the stability of the formulated compound or its delivery form. Formulations of the polymorph of the present disclosure may also contain additives for enhancement of absorption and bioavailability (e.g., vitamin E TPGS).

The pharmaceutical compositions of this disclosure may be administered orally, parenterally or via an implanted reservoir. The term parenteral as used herein includes subcutaneous, intracutaneous, intravenous, intramuscular,intra-articular, intrasynovial, intrasternal, intrathecal, and intralesional injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents and suspending agents. The details concerning the preparation of such compounds are known to those skilled in the art.

When orally administered, the pharmaceutical compositions of this disclosure may be administered in any orally acceptable dosage form including, but not limited to, capsules, tablets, and aqueous suspensions and solutions. In the case of tablets for oral use, carriers which are commonly used include lactose and corn starch. Lubricating agents, such as magnesium stearate, can also be added. For oral administration in a capsule form, useful carriers/diluents include lactose, high and low molecular weight polyethylene glycol, and dried corn starch. When aqueous suspensions are administered orally, the active ingredient is combined with emulsifying and suspending agents. If desired, certain sweetening and/or flavoring and/or coloring agents may be added.

Other suitable carriers for the above noted compositions can be found in standard pharmaceutical texts, e.g. in “Remington's Pharmaceutical Sciences”, 19th ed., Mack Publishing Company, Easton, Penn., 1995. Further details concerning the design and preparation of suitable delivery forms of the pharmaceutical compositions of the disclosure are known to those skilled in the art.

Dosage levels of between about 0.05 and about 100 milligram per kilogram (“mg/kg”) body weight per day, more specifically between about 0.1 and about 50 mg/kg body weight per day of the compounds of the disclosure are typical in a monotherapy for the prevention and/or treatment of HCV mediated disease. Typically, the pharmaceutical compositions of this disclosure will be administered from about 1 to about 3 times per day or alternatively, as a continuous infusion. Such administration can be used as a chronic or acute therapy. The amount of active ingredient that may be combined with the carrier materials to produce a single dosage form will vary depending upon the host treated and the particular mode of administration.

As the skilled artisan will appreciate, lower or higher doses than those recited above may be required. Specific dosage and treatment regimens for any particular patient will depend upon a variety of factors, including the activity of the specific compound employed, the age, body weight, general health status, gender, diet, time of administration, the duration of treatment, rate of excretion, drug combination, the severity and course of the infection, the patient's disposition to the infection and the judgment of the treating physician. In one embodiment, unit dosage formulations are those containing a daily dose or sub-dose, as herein above recited, or an appropriate fraction thereof, of an active ingredient. Generally, treatment is initiated with small dosages substantially less than the optimum dose of the peptide. Thereafter, the dosage is increased by small increments until the optimum effect under the circumstances is reached. In general, the compound is most desirably administered at a concentration level that will generally afford antivirally effective results without causing any harmful or deleterious side effects.

When the compositions of this disclosure comprise a combination of the polymorph of the disclosure and one or more additional therapeutic or prophylactic agents, both the compound and the additional agent are usually present at dosage levels of between about 10 and 100 percent, and more preferably between about 10 and 80 percent of the dosage normally administered in a monotherapy regimen. Administration of the one or more additional agents may occur prior to, after, or simultaneously with the polymorph of the present disclosure.

When the polymorph is formulated together with a pharmaceutically acceptable carrier, the resulting composition may be administered in vivo to mammals, such as man, to inhibit HCV NS3 protease or to treat or prevent HCV virus infection. Such treatment may also be achieved using the polymorph of this disclosure in combination with agents which include, but are not limited to: Immunomodulatory agents, such as interferons; other antiviral agents such as ribavirin, amantadine; other inhibitors of HCV NS3 protease; inhibitors of other targets in the HCV life cycle such as helicase, polymerase, metalloprotease, or internal ribosome entry site; or combinations thereof. The additional agents may be combined with the polymorph of this disclosure to create a single dosage form. Alternatively these additional agents may be separately administered to a mammal as part of a multiple dosage form.

Certain illustrative compounds having anti-HCV activity include those disclosed in the following publications: WO 02/04425 A2 published Jan. 17, 2002, WO 03/007945 Al published Jan. 30, 2003, WO 03/010141 A2 published Feb. 6, 2003, WO 03/010142 A2 published Feb. 6, 2003, WO 03/010143 A1 published Feb. 6, 2003, WO 03/000254 A1 published Jan. 3, 2003, WO 01/32153 A2 published May 10, 2001, WO 00/06529 published Feb. 10, 2000, WO 00/18231 published Apr. 6, 2000, WO 00/10573 published Mar. 2, 2000, WO 00/13708 published Mar. 16, 2000, WO 01/85172 A1 published Nov. 15, 2001, WO 03/037893 A1 published May 8, 2003, WO 03/037894 A1 published May 8, 2003, WO 03/037895 A1 published May 8, 2003, WO 02/100851 A2 published Dec. 19, 2002, WO 02/100846 A1 published Dec. 19, 2002, EP 1256628 A2 published Nov. 13, 2002, WO 99/01582 published Jan. 14, 1999, WO 00/09543 published Feb. 24, 2000.

Table 1 below lists some illustrative examples of compounds that can be administered with the compounds of this disclosure. The compounds of the disclosure can be administered with other anti-HCV activity compounds in combination therapy, either jointly or separately, or by combining the compounds into a composition.

TABLE 1

| | Type of Inhibitor or | |
|---|---|---|
| Brand Name | Target | Source Company |
| Omega IFN | IFN-ω | BioMedicines Inc., |
| | | Emeryville, CA |
| BILN-2061 | serine protease inhibitor | Boehringer Ingelheim |
| | | Pharma KG, Ingelheim, |
| | | Germany |
| Summetrel | antiviral | Endo Pharmaceuticals |
| | | Holdings Inc., Chadds |
| | | Ford, PA |
| Roferon A | IFN-α2a | F. Hoffmann-La Roche |
| | | LTD, Basel, Switzerland |
| Pegasys | PEGylated IFN-α2a | F. Hoffmann-La Roche |
| | | LTD, Basel, Switzerland |
| Pegasys and Ribavirin | PEGylated IFN- | F. Hoffmann-La Roche |
| | α2a/ribavirin | LTD, Basel, Switzerland |
| CellCept | HCV IgG | F. Hoffmann-La Roche |
| | immunosuppressant | LTD, Basel, Switzerland |
| Wellferon | lymphoblastoid IFN- | GlaxoSmithKline plc, |
| | αn1 | Uxbridge, UK |
| Albuferon - α | albumin IFN-α2b | Human Genome Sciences |
| | | Inc., Rockville, MD |
| Levovirin | ribavirin | ICN Pharmaceuticals, |
| | | Costa Mesa, CA |
| IDN-6556 | caspase inhibitor | Idun Pharmaceuticals Inc. |
| | | San Diego, CA |
| IP-501 | antifibrotic | Indevus Pharmaceuticals |
| | | Inc., Lexington, MA |
| Actimmune | INF-γ | InterMune Inc., Brisbane, |
| | | CA |
| Infergen A | IFN alfacon-1 | InterMune |
| | | Pharmaceuticals Inc., |
| | | Brisbane, CA |
| ISIS 14803 | antisense | ISIS Pharmaceuticals Inc, |
| | | Carlsbad, CA/Elan |
| | | Phamaceuticals Inc., New |
| | | York, NY |
| JTK-003 | RdRp inhibitor | Japan Tobacco Inc., |
| | | Tokyo, Japan |
| Pegasys and Ceplene | PEGylated IFN-α2a/ | Maxim Pharmaceuticals |
| | immune modulator | Inc., San Diego, CA |
| Ceplene | immune modulator | Maxim Pharmaceuticals |
| | | Inc., San Diego, CA |
| Civacir | HCV IgG | Nabi Biopharmaceuticals |
| | immunosuppressant | Inc., Boca Raton, FL |
| Intron A and Zadaxin | IFN-α2b/α1-thymosin | RegeneRx |
| | | Biopharmiceuticals Inc., |
| | | Bethesda, MD/ |
| | | SciClone Pharmaceuticals |
| | | Inc, San Mateo, CA |
| Levovirin | IMPDH inhibitor | Ribapharm Inc., Costa |
| | | Mesa, CA |
| Viramidine | IMPDH inhibitor | Ribapharm Inc., Costa |
| | | Mesa, CA |
| Heptazyme | ribozyme | Ribozyme |
| | | Pharmaceuticals Inc., |
| | | Boulder, CO |
| Intron A | IFN-α2b | Schering-Plough |
| | | Corporation, Kenilworth, |
| | | NJ |
| PEG-Intron | PEGylated IFN-α2b | Schering-Plough |
| | | Corporation, Kenilworth, |
| | | NJ |
| Rebetron | IFN-α2b/ribavirin | Schering-Plough |
| | | Corporation, Kenilworth, |
| | | NJ |
| Ribavirin | ribavirin | Schering-Plough |
| | | Corporation, Kenilworth, |
| | | NJ |
| PEG-Intron/Ribavirin | PEGylated IFN- | Schering-Plough |
| | α2b/ribavirin | Corporation, Kenilworth, |
| | | NJ |
| Zadazim | immune modulator | SciClone Pharmaceuticals |
| | | Inc., San Mateo, CA |
| Rebif | IFN-β1a | Serono, Geneva, |
| | | Switzerland |
| IFN-β and EMZ701 | IFN-β and EMZ701 | Transition Therapeutics |
| | | Inc., Ontario, Canada |
| T67 | β-tubulin inhibitor | Tularik Inc., South San |
| | | Francisco, CA |
| VX-497 | IMPDH inhibitor | Vertex Pharmaceuticals |
| | | Inc., Cambridge, MA |
| 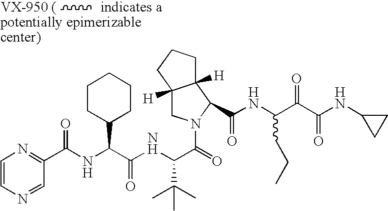 | serine protease inhibitor | Vertex Pharmaceuticals Inc., Cambridge, MA |
| Omniferon | natural IFN-α | Viragen Inc., Plantation, |
| | | FL |
| XTL-002 | monoclonal antibody | XTL Biopharmaceuticals |
| | | Ltd., Rehovot, Isreal |

Another aspect of this disclosure provides methods of inhibiting HVC NS3 protease activity in patients by administering the polymorph of the present disclosure.

In one embodiment, these methods are useful in decreasing HCV NS3 protease activity in the patient. If the pharmaceutical composition comprises only the polymorph of this disclosure as the active component, such methods may additionally comprise the step of administering to said patient an agent selected from an immunomodulatory agent, an antiviral agent, a HCV protease inhibitor, or an inhibitor of other targets in the HCV life cycle such as, for example, helicase, polymerase, or metalloprotease. Such additional agent may be administered to the patient prior to, concurrently with, or following the administration of the compounds of this disclosure.

In another embodiment, these methods are useful for inhibiting viral replication in a patient. Such methods can be useful in treating or preventing HCV disease.

The polymorph of the disclosure may also be used as a laboratory reagent. The polymorph may be instrumental in providing research tools for designing of viral replication assays, validation of animal assay systems and structural biology studies to further enhance knowledge of the HCV disease mechanisms.

The polymorph of this disclosure may also be used to treat or prevent viral contamination of materials and therefore reduce the risk of viral infection of laboratory or medical personnel or patients who come in contact with such materials, e.g., blood, tissue, surgical instruments and garments, laboratory instruments and garments, and blood collection or transfusion apparatuses and materials.

In one embodiment the polymorph of the present disclosure is formulated as a solid crystal dispersion capsule. A sample preparation is as follows:

A. Components:

Per Capsule:

1) 5 mg or 20 mg (as the free acid) of Compound (I):

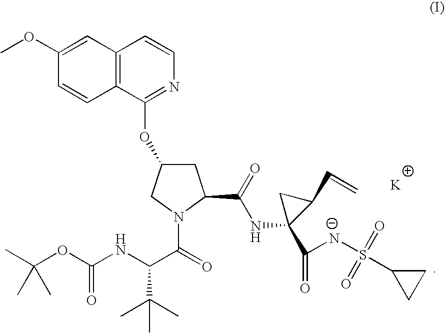

2) D-alpha tocopheryl polyethylene glycol 1000 succinate (TPGS): 365 mg 3) Hard gelatin capsule shell: Size #1

B. Process:

Per Batch:

1. Melt sufficient quantity of TPGS in a suitable container at 50° C.±5° C. until a clear liquid is obtained.
2. Transfer the required amount of TPGS from step 1 into an appropriate batching vessel maintained at 50° C.±5° C.
3. Agitate the contents of the batching vessel from step 2 to 100-1000 rpm while maintaining temperature at 50° C.±5° C.
4. Slowly add required amount of Compound of formula (III) in incremental amounts, with continuous stirring, into the batching vessel from step 3 while maintaining temperature at 50° C.±5° C. to form a suspension.
5. Continue mixing the suspension in the batching vessel from step 4 for approximately 1 hour while maintaining temperature at 50° C.±5° C.
6. Homogenize the contents of the batching vessel from step 5 to form a uniform suspension.
7. Resume mixing the suspension from step 6 for at least 1 hour while maintaining temperature at 50° C.±5° C.
8. Fill gelatin capsule shells using a suitable liquid capsule filling machine while maintaining temperature of the suspension at 50° C.±5° C. and stirring speed at 100-1000 rpm.
9. Cool capsules to room temperature.

The following non-limiting examples are illustrative of the disclosure.

### EXAMPLES

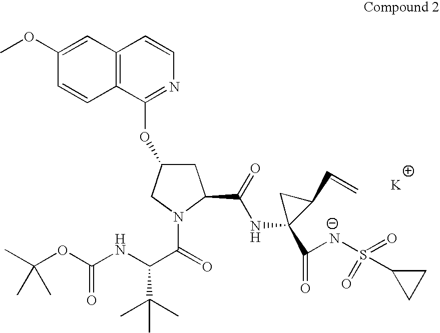

Compound 2 was prepared according to the procedure described in U.S. Pat. Ser. No. 6,995,174.

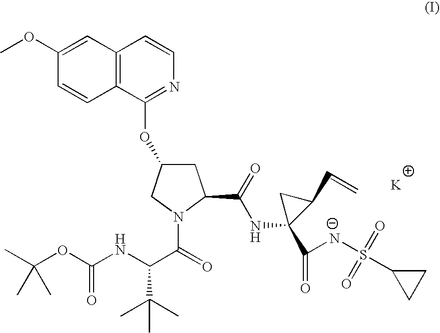

To a 25 ml 2 neck flask was added a stir bar, septa and N₂ gas adapter. Compound 2 (99.7 mg, 0.140 mmol) was weighed out and added to the reaction flask. The reaction flask was purged and placed under a N₂ atmosphere. 850 μl of acetone was added to the flask to provide a clear solution. To this solution at room temperature was added 780 μl of a 0.1 79M solution of KOH (aq.) prepared by the dissolution of solid KOH (502.8 mg, 8.97 mmol) in 50 ml of H₂O. The solution warmed slightly upon addition of the KOH but remained clear. The clear solution was allowed to stir at room temperature for 2 hours. The product crystallized out of solution and was isolated by filtration. The cake was washed with cold acetone to afford 42 mg (40% yield) of the desired product as fine white needles.

### Alternative Preparation of Compound (I)

In a 500 mL Erlenmeyer flask with magnetic stirbar, Compound 2 (49.58 g) was dissolved in acetone (250 mL) to give a clear nearly colorless solution. This solution was filtered through Whatman 1 paper into a 500 mL round bottom flask equipped with with a mechanical stirrer, addition funnel, and temperature probe. The filter was washed with an additional 50 mL of acetone. The solution was heated to 45° C. and treated with a solution of potassium hydroxide (4.54 g) in water (70.00 mL). After 10 mL of base had been added over 5 minutes, the reaction mixture began to crystallize without seeding. The base addition was paused at the 15 mL mark, and within 30 minutes the crystallization was well initiated. The remaining base was added dropwise at 45° C., with a total addition time of 3 hours including the pause. The reaction mixture was heated to reflux and acetone was removed by distillation (180 mL with a head temperature of 56-59° C.). Water (100 mL) was added to the reaction mixture over 1 hour, while the reaction mixture was cooled to 35° C. The mixture was further cooled to 14° C. over ˜70 minutes. The thick slurry was filtered through Whatman 1 paper, washed with 200 mL cold (acetone/ water 1:1) and dried on the funnel under nitrogen then under high vacuum at room temperature for 36 hours. The product wt. was 48.44 g as a white solid. With an in-process AP of 99.34 @ 220 nm. Product was submitted for form determination and found to be N-1 by XRD, DSC and TGA.

This crystalline form was analyzed using one or more of the testing methods described below.

1 Single Crystal X-Ray Measurements

A Bruker SMART 2K CCD diffractometer equipped with graphite-monochromated Cu Kα radiation, (λ=1.54056 Å) was used to collect diffraction data at the room temperature. A full data set was collected using the ω scan mode over the 2θ range with a crystal-to-detector distance of 4.98 cm. An empirical absorption correction utilized the SADABS routine associated with the diffractometer (Bruker AXS. 1998, SMART and SAINTPLUS. Area Detector Control and Integration Software, Bruker AXS, Madison, Wis. USA). The final unit cell parameters were determined using the entire data set.

The structure was solved by direct methods and refined by the full-matrix least-squares techniques, using the SHELXTL software package (Sheldrick, GM. 1997, SHELXTL. Structure Determination Programs. Version 5.10, Bruker AXS, Madison, Wis. USA.). The function minimized in the refinements was Σ_{W}(|F_{O}|−|F_{C}|)². R is defined as Σ∥F_{O}|−|F_{C}∥/Σ|F_{O}| while R_{W}=[Σ_{W}(|F_{O}|−|F_{C}|)2/Σ_{w}|F_{O}|²]^{1/2}, where w is an appropriate weighting function based on errors in the observed intensities. Difference Fourier maps were examined at all stages of refinement. All non-hydrogen atoms were refined with anisotropic thermal displacement parameters. The hydrogen atoms associated with hydrogen bonding were located in the final difference Fourier maps while the positions of the other hydrogen atoms were calculated from an idealized geometry with standard bond lengths and angles. They were assigned isotropic temperature factors and included in structure factor calculations with fixed parameters.

The crystal data of the N-1 form is shown in Table 2. The fractional atomic coordinates are listed in Table 3. Each of the atoms (except H) in form N-1 is labeled according to FIG. 4.

TABLE 2
Crystal Data of Form N-1

| | |
|---|---|
| Temperature | 293(2) K |
| Wavelength | 1.54178 \acute{Å} |
| Crystal system, space group | Orthorhombic, P2₁2₁2₁ |
| Unit cell dimensions | a = 6.2239(1) \acute{Å} alpha = 90° |
| | b = 20.9360(3) \acute{Å} beta = 90° |
| | c = 29.1860(5) \acute{Å} gamma = 90° |
| Volume | 3803.04(10) \acute{Å}³ |
| Z, Calculated density | 4, 1.313 Mg/m³ |
| Absorption coefficient | 2.224 mm⁻¹ |
| F(000) | 1592 |
| Crystal size | 0.55 × 0.12 × 0.03 mm |
| Theta range for data collection | 2.60 to 65.24 deg. |
| Limiting indices | −6 <= h <= 6, −21 <= k <= 24, |
| | −33 <= 1 <= 32 |
| Reflections collected/unique | 20213/6273 [R(int) = 0.0575] |
| Completeness to theta = 65.24 | 96.7 percent |
| Absorption correction | SADABS |
| Max. and min. transmission | 1.000 and 0.781 |
| Refinement method | Full-matrix least-squares on F\hat{ }2 |
| Data/restraints/parameters | 6273/0/462 |
| Goodness-of-fit on F² | 1.007 |
| Final R indices [I > 2sigma(I)] | R1 = 0.0422, wR2 = 0.1044 |
| R indices (all data) | R1 = 0.0502, wR2 = 0.1084 |
| Absolute structure parameter | 0.014(11) |
| Largest diff. peak and hole | 0.271 and −0.172 e · A⁻³ |

TABLE 3
Atomic coordinates (×10⁴) and equivalent isotropic displacement
parameters (A² × 10³) for Form N-1. U(eq) is defined as one third
of the trace of the orthogonalized Uij tensor.

| | x | y | z | U(eq) |
|---|---|---|---|---|
| K(1) | 8737(1) | 2063(1) | 5282(1) | 63(1) |
| S(1) | 8675(1) | 3810(1) | 5049(1) | 51(1) |
| O(1) | −634(3) | 4675(1) | 3011(1) | 58(1) |
| O(2) | 4409(4) | 3144(1) | 2508(1) | 92(1) |
| O(3) | 1083(4) | 2829(1) | 2280(1) | 73(1) |
| O(4) | 3688(3) | 3719(1) | 3950(1) | 65(1) |
| O(5) | 9467(3) | 2784(1) | 4458(1) | 60(1) |
| O(6) | 10960(3) | 3799(1) | 5127(1) | 68(1) |
| O(7) | 7446(4) | 3342(1) | 5306(1) | 66(1) |
| O(8) | 2483(3) | 6034(1) | 4156(1) | 64(1) |
| O(9) | −817(5) | 8888(1) | 4075(1) | 94(1) |
| N(1) | 2296(4) | 4803(1) | 3450(1) | 50(1) |
| N(2) | 1593(4) | 3826(1) | 2479(1) | 60(1) |
| N(3) | 6980(4) | 3829(1) | 3622(1) | 49(1) |
| N(4) | 8052(4) | 3802(1) | 4521(1) | 52(1) |
| N(5) | −576(5) | 5923(1) | 4591(1) | 67(1) |
| C(1) | 4562(4) | 4711(1) | 3575(1) | 50(1) |
| C(2) | 4890(5) | 5191(2) | 3967(1) | 68(1) |
| C(3) | 2650(5) | 5344(1) | 4161(1) | 59(1) |
| C(4) | 1029(5) | 5042(2) | 3841(1) | 59(1) |
| C(5) | 1345(5) | 4632(1) | 3058(1) | 50(1) |
| C(6) | 2706(5) | 4376(1) | 2663(1) | 55(1) |
| C(7) | 3164(6) | 4890(2) | 2285(1) | 73(1) |
| C(8) | 4224(7) | 5478(2) | 2502(1) | 100(1) |
| C(9) | 4711(8) | 4593(3) | 1940(1) | 119(2) |
| C(10) | 1087(7) | 5085(2) | 2039(1) | 103(2) |
| C(11) | 2541(6) | 3256(2) | 2430(1) | 65(1) |
| C(12) | 1622(7) | 2145(2) | 2226(1) | 80(1) |
| C(13) | 3249(9) | 2085(2) | 1835(2) | 113(2) |
| C(14) | 2395(9) | 1876(2) | 2672(1) | 109(2) |
| C(15) | −495(9) | 1861(2) | 2097(2) | 116(2) |
| C(16) | 5008(4) | 4031(1) | 3733(1) | 47(1) |
| C(17) | 7950(4) | 3256(1) | 3798(1) | 50(1) |
| C(18) | 7308(6) | 2637(1) | 3559(1) | 65(1) |
| C(19) | 9438(6) | 2920(2) | 3472(1) | 69(1) |
| C(20) | 7107(8) | 2014(2) | 3802(1) | 86(1) |
| C(21) | 5777(14) | 1603(2) | 3736(2) | 163(3) |
| C(22) | 8561(4) | 3265(1) | 4294(1) | 49(1) |
| C(23) | 7733(5) | 4563(2) | 5206(1) | 62(1) |
| C(24) | 5426(6) | 4644(2) | 5316(1) | 81(1) |
| C(25) | 7056(7) | 4663(2) | 5693(1) | 85(1) |
| C(26) | 724(5) | 6292(2) | 4360(1) | 56(1) |
| C(27) | 437(5) | 6962(1) | 4301(1) | 56(1) |
| C(28) | 1875(6) | 7361(2) | 4063(1) | 71(1) |
| C(29) | 1388(7) | 7994(2) | 3999(1) | 80(1) |
| C(30) | −529(6) | 8255(2) | 4166(1) | 71(1) |
| C(31) | −1945(6) | 7883(2) | 4403(1) | 67(1) |
| C(32) | −1486(5) | 7230(1) | 4475(1) | 58(1) |
| C(33) | −2875(6) | 6827(2) | 4718(1) | 68(1) |
| C(34) | −2358(6) | 6214(2) | 4774(1) | 76(1) |
| C(35) | −2799(7) | 9163(2) | 4216(2) | 98(1) |

2. Powder X-Ray Diffraction

X-Ray powder diffraction (PXRD) data were obtained using a Bruker D8 Advance GADDS system. Powder samples were placed in thin walled glass capillaries; the capillary was rotated during data collection. The sample-detector distance was 15 cm. The radiation was Cu Kα (λ=1.5418 Å). Data were collected for 3<2θ <35° with a sample exposure time of at least 1800 seconds.

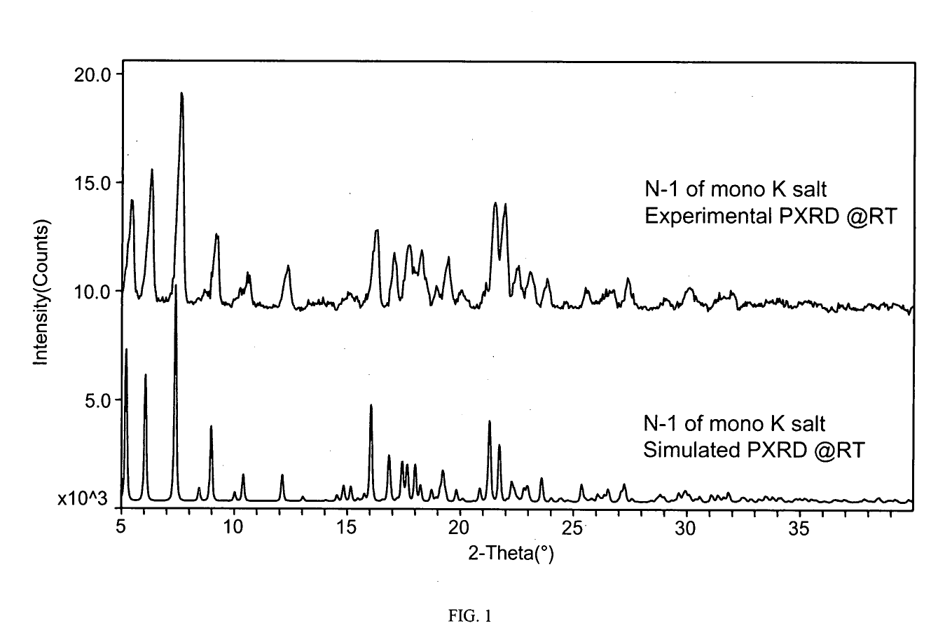

The results of the PXRD pattern and a simulated pattern calculated from the single crystal data are shown in FIG. 1.

Table 4 lists the selected PXRD peaks that describe Form N-1 of Compound (I).

TABLE 4
Positions (degrees in 2θ) of Selected PXRD Peaks
Form N-1

5.2
6.1
7.4
8.4
9.0
10.0
10.4
12.1
16.0
16.8

3. Differential Scanning Calorimetry

Differential scanning calorimetry was conducted using a TA Instruments™ model Q1000 or 2920. The sample (about 2-6 mg) was weighed in an open aluminum pan or sealed pan with pin hole and recorded accurately to a hundredth of a millirgam, and transferred to the DSC. For each analysis, the DSC cell/sample chamber was purged with 50 ml/min of ultra-high purity nitrogen gas. The instrument was calibrated with high purity indium. The heating rate was 10° C. per minute in the temperature range between 25 and 300° C. The heat flow, which was normalized by sample weight, was plotted versus the measured sample temperature. The data were reported in units of watts/gram (“W/g”). The plot was made with the endothermic peaks pointing down. The endothermic melt peak (melting point) was evaluated for extrapolated onset temperature.

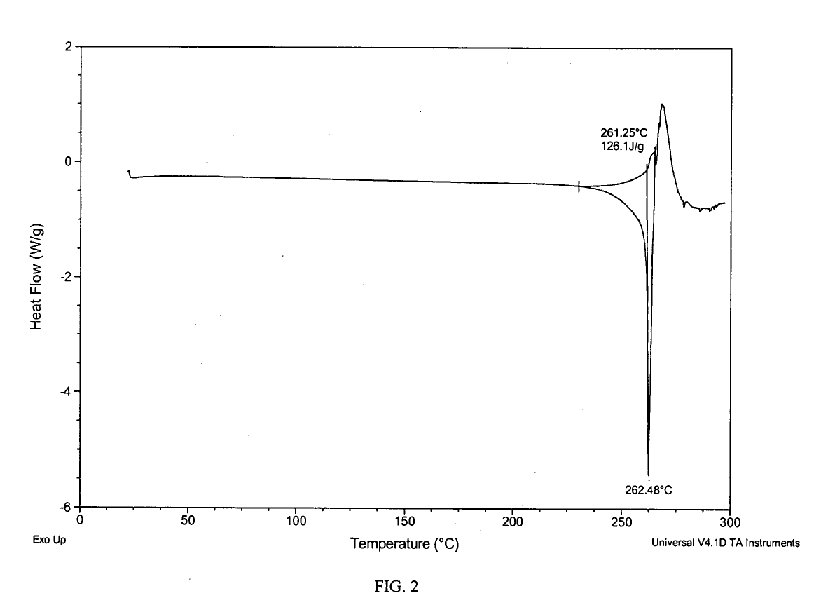

The results are shown in FIG. 2.

4. Thermogravimetric Analysis (TGA) (Open Pan)

Thermal gravimetric analysis (TGA) experiments were performed in a TA Instruments™ model Q500 or 2950. The sample (about 10-30 mg) was placed in a platinum pan. The weight of the sample was measured accurately and recorded to a thousand of a milligram by the instrument. The furnace was purged with nitrogen gas at 100 mL/min. Data were collected between room temperature and 300° C. at 10° C./min heating rate.

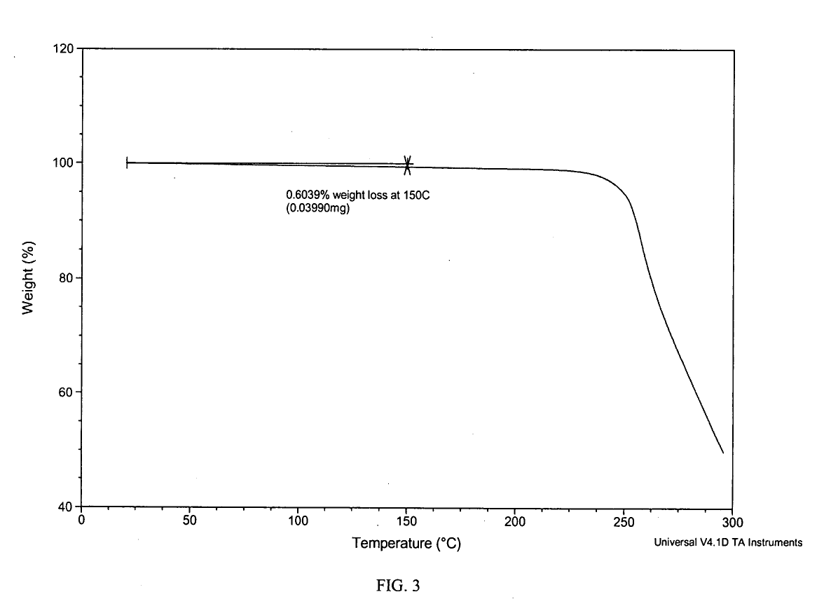

The results are shown in FIG. 3.

What is claimed is:

1. A crystalline form of (1R,2S)-N-[(1,1-dimethylethoxy)carbonyl]-3-methyl-L-valyl-(4R)-4-[(6-methoxy-1-isoquinolinyl)oxy]-L-prolyl-1-amino-N-(cyclopropylsulfonyl)-2-ethenyl-cyclopropanecarboxamide or 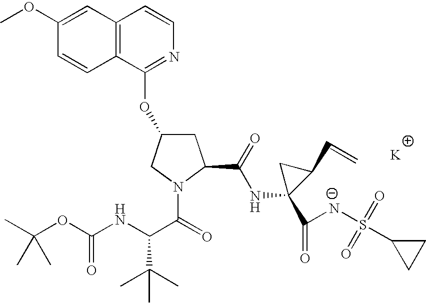
comprising Form N-1.

2. The crystalline form of claim 1 consisting essentially of Form N-1.

3. The crystalline form of claim 1 wherein said Form N-1 has a purity of at least 90 weight percent.

4. The crystalline form of claim 1 wherein said Form N-1 has a purity of at least 95 weight percent.

5. The crystalline form of claim 1 wherein said Form N-1 has a purity of at least 99 weight percent.

6. The crystalline form of claim 1 characterized by unit cell parameters substantially equal to the following:
Cell dimensions: a=6.2239 Å
b=20.9360 Å
c=29.1860 Å
α=90 degrees
β=90 degrees
γ=90 degrees
Space group P2₁2₁2₁
Molecules/unit cell 4
wherein measurement of said crystalline form is at a temperature between about 20° C. to about 25° C.

7. The crystalline form of claim 1 characterized by fractional atomic coordinates within the unit cell substantially as listed in Table 3.

8. The crystalline form of claim 1 characterized by a powder X-Ray diffraction pattern comprising four or more 20θ values (CuKα λ=1.5418 Å) selected from the group consisting of 5.2, 6.1, 7.4, 8.4, 9.0, 10.0, 10.4, 12.1, 16.0, and 16.8 at a temperature between about 20° C. and about 25° C.

9. The crystalline form of claim 8 further characterized by a powder X-Ray diffraction pattern comprising five or more 20θ values (CuKα λ=1.5418 Å) selected from the group consisting of 5.2, 6.1, 7.4, 8.4, 9.0, 10.0, 10.4, 12.1, 16.0, and 16.8 at a temperature between about 20° C. and about 25° C.

10. The crystalline form of claim 1 characterized by one or more of the following:
a) a unit cell with parameters substantially equal to the following:
Cell dimensions: a=6.2239 Å
b=20.9360 Å
c=29.1860Å
α=90 degrees
β=90 degrees
γ=90 degrees
Space group P2₁2₁2₁
Molecules/unit cell 4
wherein measurement of said crystalline form is at a temperature between about 20° C. and about 25° C.;
b) a powder X-Ray diffraction pattern comprising four or more 20θ values (CuKα λ=1.5418 Å) selected from the group consisting of 5.2, 6.1, 7.4, 8.4, 9.0, 10.0, 10.4, 12.1, 16.0, and 16.8 at a temperature between about 20° C. and about 25° C.; and/or
c) a melting point in the range of about 252° C. to about 262° C.

11. A pharmaceutical composition comprising the crystalline form of claim 1 and a pharmaceutically acceptable carrier or diluent.

12. The pharmaceutical composition of claim 11 wherein said Form N-1 has a purity of at least 90 weight percent.

13. The pharmaceutical composition of claim 11 wherein said Form N-1 has a purity of at least 95 weight percent.

14. The pharmaceutical composition of claim 11 wherein said Form N-1 has a purity of at least 99 weight percent.

15. A pharmaceutical composition comprising the crystalline form of claim 1 in combination with a second compound having anti-HCV activity.

16. The pharmaceutical composition of claim 15 wherein said Form N-1 has a purity of at least 90 weight percent.

17. The pharmaceutical composition of claim 15 wherein said Form N-1 has a purity of at least 95 weight percent.

18. The pharmaceutical composition of claim 15 wherein said Form N-1 has a purity of at least 99 weight percent.

19. The composition of claim 15 wherein the second compound having anti-HCV activity is an interferon.

20. The composition of claim 19 wherein the interferon is selected from interferon alpha 2B, pegylated interferon alpha, consensus interferon, interferon alpha 2A, and lymphoblastiod interferon tau.

21. The composition of claim 15 wherein the second compound having anti-HCV activity is selected from interleukin 2, interleukin 6, interleukin 12, a compound that enhances the development of a type 1 helper T cell response, interfering RNA, anti-sense RNA, Imiqimod, ribavirin, an inosine 5′-monophospate dehydrogenase inhibitor, amantadine, and rimantadine.

22. A method of treating HCV infection in a mammal comprising administering to the mammal a therapeutically-effective amount of the crystalline form of (1R,2S)-N-[(1,1-dimethylethoxy)carbonyl]-3-methyl-L-valyl-(4R)-4-[(6-methoxy-1-isoquinolinyl)oxy]-L-prolyl-1-amino-N-(cyclopropylsulfonyl)-2-ethenyl-cyclopropanecarboxamide of claim 1.

23. The method of claim 22 wherein said Form N-1 has a purity of at least 90 weight percent.

24. The method of claim 22 wherein said Form N-1 has a purity of at least 95 weight percent.

25. The method of claim 22 wherein said Form N-1 has a purity of at least 99 weight percent.

26. The method of claim 22 wherein the mammal is a human.

27. A composition comprising at least 90 weight percent of the crystalline form of claim 1, based the weight of the composition.
